(19) United States
(12) Patent Application Publication
(10) Pub. No.: US 2007/0244318 A1
(43) Pub. Date: Oct. 18, 2007

(54) Process for the Preparation of Levofloxacin Hemihydrate

(76) Inventors: Davuluri Rammohan Rao, Hyderabad (IN); Shiprakash Dhar Dwivedi, Hyderabad (IN); Pamujula Sreenivasulu, Hyderab (IN); Arabinda Sahu, Hyderabad (IN); Ganala Naga Trinadhachari, Hyderabad (IN); Surapaneni Sasi Kiran, Hyderabad (IN)

Correspondence Address: MOORE & VAN ALLEN PLLC, P.O. BOX 13706, Research Triangle Park, NC, 27709, US

(21) Appl. No.: 10/578,078

(22) PCT Filed: Aug. 11, 2004
(86) PCT No.: PCT/IN04/00343

§ 371 Date: Mar. 7, 2007

(51) Int. Cl. C07D 491/14

(52) U.S. Cl. 544/101

(57) ABSTRACT

The present invention relates to an improved process for preparation of Levofloxacin hemihydrate having single individual impurity not more than 0.1% and free from particulate matter & from the other enantiomer (R-form) which comprises dissolving levofloxacin technical grade in aqueous alkaline solution, treating the resulting solution with activated carbon at room temperature, removing the undissolved particulate matter filtration, bringing the pH of the aqueous alkaline levofloxacin solution to neutral using dilute mineral acid, removing the precipitated particulate matter by filtration, acidifying the resulting solution, treating the acidified solution with activated carbon at room temperature, filtering the undissolved particulate matter by filtration, neutralizing the acidic solution, filtering again to remove any particulate matter present and, extracting the resulting product with chlorinated solvent and concentrating under vacuum using aqueous tetrahydrofuran or in admixture with other organic solvents to get highly pure levofloxacin hemihydrate having single individual impurity is less than 0.1% and free from particulate matter & from the other enantiomer (R-form).

### FIELD OF INVENTION

This invention provides an improved process for the preparation of Levofloxacin hemihydrate. More specially, the invention provides an improved process for the preparation of high purity Levofloxacin hemihydrate from Levofloxacin technical. The Levofloxacin hemihydrate prepared has a single individual impurity less than 0.1% free from particulate matter and other enantiomer (R-form). Levofloxacin hemihydrate prepared by the process of the present invention is useful as an antibacterial. The invention also relates to an improved process for the preparation of Levofloxacin hemihydrate starting from 2,3,4,5,tetrafluoro benzoic acid through the intermediates Levofloxacin Q acid and Levofloxacin technical.

### BACKGROUND OF THE INVENTION

Levofloxacin is the S-(−) isomer of the fluoroquinoline antibacterial Ofloxacin. Levofloxacin is generally considered to be about twice as active as Ofloxacin. It has a broad spectrum of activity, which included Gram-positive bacteria. [Davis R, Bryson H M, Drugs, 4, 677(1994)]. Levofloxacin is given by mouth or intravenously for the treatment of susceptible infections in a usual dose of 250 or 500 mg. once or twice daily. It is also administered topically as 0.5% eye drops for the treatment of bacteria conjunctivitis [Martindale, The Complete Drug Reference, 33^{rd} edition, pp. 219 (2002) and references cited therein].

The Levofloxacin hemihydrate is (S)-9-Fluoro-2,3-dihydro-3-methyl-10-(4-methyl-1piperazinyl)-7-oxo-7H-pyrido[1,2,3-de]-1,4-benzoxazine-6-carboxylic acid hemihydrate. The chemical structure of Levofloxacin hemihydrate (CAS Registry No. [138199-71-0]) is shown as formula I.

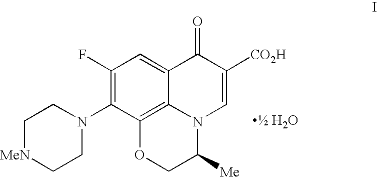

U.S. Pat. No. 5,545,737 and EP patent No. 0444678 disclose a process for the preparation of levofloxacin hemihydrate or monohydrate selectively by controlling the water content of an aqueous solvent selected from the group consisting of methanol, ethanol, propanol and acetone in which levofloxacin is dissolved during crystallization.

Niddamhil Desheim, Valeric et al. disclose in WO 03/045329, and in WO 03/028665, methods for the purification of Levofloxacin using polar solvent such as DMSO, methyl ethyl ketone, acetonitrile, butanol and mixtures thereof and aqueous mixture thereof and/or using an antioxidant.

Levofloxacin was first disclosed in U.S. Pat. No. 5,053,407 and in Antimicrob. Agents chemother. 29, 163 (1986) by Hayakawa I, et al. In this process 9,10-Difluoro-3-(hydroxymethyl)-7-oxo-2,3-dihydro-7H-pyrido[1,2,3-fi?e]-1,4-benzoxazine-6-carboxylic acid ethyl ester, a racemic intermediate in the synthesis of Ofloxacin was resolved and esterified with 3,5-dinitro benzoyl chloride, separated by HPLC column SUMIPAX-OA-4200, using hexane, 1,2-dichloro ethane, ethanol as carrier solvent. The (−) optical isomer is partially hydrolyzed with ethanolic aqueous sodium bicarbonate to afford the (−) alcohol, which is treated with triphenyl phosphite methiodide in DMF giving the corresponding (−)-iodomethyl derivative. The reduction and simultaneous hydrolysis with tributyltin hydride in ethanol yield (−)-9,10-difluoro-3-methyl-7-oxo-2,3-dihydro-7H-pyrido[1,2,3-Je]1,4-benzoxazine-6-carboxylic acid which is finally treated with N-methyl piperazine at 120° C. in DMSO to give (−) Ofloxacin i.e. Levofloxacin.

In U.S. Pat. No. 5,053,407 racemic-3-acetoxy methyl-7,8-difluoro-2,3-dihydro-4H-[1,4]-benzoxazine was resolved through its dinitrobenzoyl derivatives (or condensed with a cyclic amino acid or a reactive derivative through amide linkage) followed by debenzoylation, deacylation and dehydroxylation to obtained optically active 7,8-difluoro-2,3-dihydro-3-methyl-4H-[1,4]-benzoxazine which on condensation with diethyl ethoxy acrylomalonate followed by cyclization, hydrolysis and condensation with N-methyl piperazine to obtain Levofloxacin.

Scientists of Daiichi prepared the intermediate (−)9,10-difluoro-3-methyl-7-oxo-2₅3-dihydro-7H-pyrido[1,2,3-de]-1,4-benzoxazine-6-carboxylic acid by either resolution or chiral specific synthesis. While condensation with N-methyl piperazine major reports disclosed in the presence of DMSO at above 100° C. or chilation with BF₃—OEt₂ in ether and then condensation with N-methyl piperazine in the presence of DMSO, triethylamine has reported.

The preparation of Levofloxacin has also been reported in U.S. Pat. No. 4,777,253 and J. Med. Chem. 30, 2283 (1987) by Mitcher et al. Levofloxacin synthesized from 2,3,4,5-tetrafluoro benzoic acid which is treated with thionyl chloride to produce the corresponding acid chloride. Displacement of the acid chloride with malonic acid half ester in the presence of n-Butyl lithium yield the β-ketoester. The β-ketoester is then treated with a trialkyl orthoformate in the presence of an acid anhydride yielding Ethyl-2-(2,3,4,5-tetrafluoro benzoyl)-3-alkoxy acylate, which on reaction with (S)-2-amino-1-propanol to obtain enamino ketoester. The enamino ketoester is then cyclized such as by treatment with two moles equivalents of an alkali metal hydride, alkoxide at an elevated temperature to obtain alkyl-(−)-9,10-difluoro˜3-methyl-7-oxo-2,3-dihydro-7H-pyrido-[1,2,3-afe]-1,4-benzoxazine-6-carboxylate. The condensation reaction between N-methyl piperazine and alkyl (−)-9,10-difluoro-3-methyl-7-oxo-2,3-dihydro-7H-pyrido[1,2,3-fe]1,4-benzoxazine-6-carboxylate at temperature from 20° C. to 200° C. and preferably from 40° C. to 90° C. in the presence of a suitable organic solvent such as pyridine, chloroform, dimethylsulphoxide, sulfolane, dimethylformamide, dimethylacetamide, 1-methyl-2-Pyrrolidone. It is desirable to carry out the reaction in the presence of an acid-acceptor such as triethyl amine, potassium carbonate and the like a molar ratio of 1.0 to 1.2 mole of the acid-acceptor per mole of the ester. N-methyl piperazine can also be used as an acid-acceptor in which two or more molar excess is used.

Antons, Stefan et al. Bayer. A.G. have reported in German patent no DE 4428020A, a process for the preparation of (−)-9,10-difluoro-2,3-dihydro-(S)-3-methyl-7-oxo-7H-pyrido-[1,2,3-de]-1,4-benzoxazine-6-carboxylic acid starting from 2,3,4,5-tetrafluoro benzoyl chloride. The process comprises condensing 2,3,4,5-tetrafluoro benzoyl chloride with either 3-(N-methyl piperazinyl)-acetylic acrylic acid ethyl ester or 3-(N,N-dimethylamino)acetylic acrylic acid ethyl ester followed by transamination with (S)-2-Amino propanol, cyclization and hydrolysis.

Schriewer et al. Bayer A.G. has disclosed in U.S. Pat. No. 5,237,060 a process of preparing enantiomerically pure 1,8-bridged-4-quinolone-3-carboxylic acids, starting from 3-ethoxy-2-(2,3,4,5-tetrafluoro benzoyl) acrylic acid ethyl ester. 3-ethoxy-2-(2,3,4,5-tetrafluoro benzoyl) acrylic acid ethyl ester on condensation with (S)-2-amino propanol, cyclization using potassium carbonate, hydrolysis and condensation with N-methylpiperazine provides Levofloxacin.

Kim et al. of Korea Institute of Science and Technology reported in U.S. Pat. No. 5,539,110 a method for the preparation of (−) piperazine derivative. The (−) 9,10-Difluoro-2₅3-dihydro-3-methyl-7-oxo-7/?pyrido[1,2,3ˆ]-1,4-benzoxazine-6-carboxylic acid is prepared starting from (S) (+)-2-Amino-1-propanol in four steps by condensing with alkyl silylated piperazine in the presence of organic polar solvent such as pyridine, dimethylsulfoxide, acetonitrile, dimethylformamide, N-methyl pyrrolidine and sulfolane.

Juan. C. Carretero et al. in Heterocycles vol 51, No 7, 1999; have also published an efficient synthesis of Ofloxacin and Levofloxacin from 3,4-difluoroaniline. Key steps in the synthetic sequence are the regioselective functionalization at either C-2 or C-3 of the N-(tert-butoxy carbonyl)-3,4,-difluoroaniline and the construction of the benzoxazine skeleton by O-alkylation of the corresponding phenol with propylene oxide, which was transformed into Ofloxacin or Levofloxacin by condensing with N-methyl piperazine. Shohgo Atarashi et al. in Chem. Pharm. Bull 35 (5) 1896-1902 (1987). discloses, two optically active isomers of Ofloxacin and their fluoromethyl derivatives were prepared via their optically active intermediates resolved by used of high performance liquid chromatography (HPLC). The isomers were also obtained efficiently by an alternative route via separation of the diastereo isomers prepared in the reaction of benzoxazine with L-proline chloride, then condensation of N-methyl piperazine with (−)-9,10-difluoro-2,3-dihydro-(S)-3-methyl-7-oxo-7i/-pyrido-[1,2,3-de]-1,4-benzoxazine-6-carboxylic acid in the presence of dimethyl sulphoxide.

Carretero Gonzalver et al. of in U.S. Pat. No. 5,521,310 disclose a process for the preparation of benzoxazines which is to be used for the synthesis of Levofloxacin Ofloxacin and derivatives starting from 3,4-difluoroaniline.

Nakamura, Hiroshi et al. in JP patent no 2001 31,654. according to the process disclosed in this patent Ethyl 6,7,8-trifluoro-1,4-dihydro-1-(1-acetoxymethyl)ethyl-4-oxoquinoline-3-carboxylate is prepared and condensed with 1-methyl piperazine in toluene at 100° C. for 2 hrs and then cyclized in the presence of sodium hydroxide in 2-propanol at 100° C. for 2 hrs.

Levofloxacin disclosed recently in Zhonggno Yaowu Huaxue Zazhi 2000, 10 (4), 276-278 (Ch) by Li, Jiaming et al. from 2,3,4,5-tetrafluoro benzoic acid by chlorination, condensation with di-Et malonate, partial hydrolysis, decarboxylation, condensation with triethyl orthoformate, substitution with (S)-(+)-2-aminopropanol, cyclization, hydrolysis and substitution with N-methyl piperazine.

In view of the need for the Levofloxacin hemihydrate having single individual impurity not more than 0.1% and free from particulate matter for pharmaceutical applications, we under took R&D work towards this direction.

Accordingly, the main objective of the present invention is to provide an improved process for the preparation of Levofloxacin hemihydrate having single individual impurity not more than 0.1% and free from particulate matter & from the other enantiomer (R-form).

Yet another objective of the present invention is to provide an improved process for the preparation of Levofloxacin hemihydrate by dissolving Levofloxacin in water at different pH followed by filtration and using aqueous tetrahydrofuran for making the Levofloxacin hemihydrate.

Still another objective of the present invention is to provide an improved process for the preparation of Levofloxacin hemihydrate starting from 2,3,4,5/tetrafluoro benzoic acid through the intermediates Levofloxacin Q-acid and Levofloxacin technical.

### SUMMARY OF THE INVENTION

Accordingly, the present invention provides an improved process for preparation of Levofloxacin hemihydrate having single individual impurity not more than 0.1% and free from particulate matter & from the other enantiomer (R-form) which comprises
(i) dissolving levofloxacin technical grade in aqueous alkaline solution,
(ii) treating the resulting solution with activated carbon at room temperature,
(iii) removing the undissolved particulate matter through filtration,

(iv) bringing the pH of the aqueous alkaline levofloxacin solution to neutral using dilute mineral acid,
(v) removing the precipitated particulate matter by filtration,
(vi) acidifying the resulting solution,
(vii) treating the acidified solution with activated carbon at room temperature,
(viii) filtering the undissolved particulate matter by filtration,
(ix) neutralizing the acidic solution,
(x) filtering again to remove any particulate matter present and
(xi) extracting the resulting product with chlorinated solvent and concentrating under vacuum using aqueous tetrahydrofuran or in admixture with other organic solvents to get highly pure levofloxacin hemihydrate having single individual impurity less than 0.1% and free from particulate matter & from the other enantiomer (R-form).

In a preferred embodiment of the present invention the filtration in steps (viii) & (x) may be effected using a 0.2 micron filter.

Levofloxacin technical is prepared either by using pyridine as a solvent or without using solvent by any known methods. Levofloxacin technical may be stirred in water and pH is adjusted to 8.0 to 12.0, preferably 10.0-12.0, more preferably 11.0-11.5 with dilute alkali metal hydroxide solution, preferably 5 to 20% alkali solution, more preferably 8-10%. The alkali metal hydroxide which may be used may be either sodium hydroxide or potassium hydroxide, preferably sodium hydroxide. The aqueous alkaline levofloxacin solution is treated with activated carbon, and the clear solution is filtered. Then the pH is brought to 7.0-7.5 using dilute hydrochloric acid, preferably 0.5N to 5N hydrochloric acid, more preferably 1N hydrochloric acid. The precipitated particulate matter is filtered. Again the aqueous solution pH may be adjusted to 3.0-6.0 preferably 4.0 to 5.5 more preferably 4.5-5.0 using glacial acetic acid. The aqueous acidic levofloxacin solution was treated with activated carbon at room temperature and the clear solution may be filtered preferably through micron filter. Finally the pH of the aqueous solution may be adjusted to neutral preferably 7.0-7.5 using dilute aqueous ammonia solution. The neutral aqueous solution may be again filtered preferably through micron filter and extracted with chlorinated solvent preferably methylene chloride. The extract may be concentrated under vacuum (600-650 mm of Hg) below 40° C. The resulting residue was concentrated after stirring with tetrahydrofuran or its mixture any other organic solvent. Finally the residue was slurred with 1-5% aqueous tetrahydrofuran preferably with 2-2.5% aqueous tetrahydrofuran at 40-70° C. preferably at 50-60° C. more preferably at 58-60° C. for 30 minutes to 2 hours preferably 30 minutes to 1 hour then cooled to −5 to 15° C. preferably 0-5° C. and stirred for 30 minutes to 2 hours preferably 1 hour to 1 hour 30 minutes. The product was filtered and suck dried for 15 minutes to 1 hour preferably 30 minutes to 45 minutes and the product was dried at 50-80° C. preferably at 70-75° C. for 2 to 7 hours preferably 4-6 hours more preferably 5 to 5 hours 30 minutes to give highly pure Levofloxacin hemihydrate with single individual impurity less than 0.1%. Elemental analysis calculated for C₁₈H₂₀FN₃O₄.½H₂O is C 58.37%, H 5.71%, N 11.35%, and found is C 58.38%, H 5.67%, N 11.38%.

The X-ray diffraction pattern of the Levofloxacin hemihydrate prepared by the process of the present invention is found to be identical to that of the Levofloxacin hemihydrate prepared by the process reported in U.S. Pat. No. 5, 545,737, EP patent 0444678 and Chem. Pharm. Bull, 43 (4) 649-653 (1995). The typical X-ray diffraction pattern of the prepared Levofloxacin hemihydrate is given below.

| Angle | d value | Intensity |
|---|---|---|
| 2-Theta⁰ | angstrom | % |
| 6.654 | 13.27346 | 100.0 |
| 9.704 | 9.10667 | 60.2 |
| 13.092 | 6.75695 | 38.5 |
| 13.360 | 6.62202 | 4.1 |
| 13.761 | 6.42997 | 2.2 |
| 14.744 | 6.00343 | 1.0 |
| 15.833 | 5.59291 | 7.9 |
| 18.382 | 4.82260 | 1.3 |
| 19.918 | 4.68723 | 4.8 |
| 19.435 | 4.56359 | 13.0 |
| 20.037 | 4.42795 | 9.4 |
| 23.442 | 3.79187 | 1.6 |
| 24.222 | 3.67147 | 1.3 |
| 24.459 | 3.63649 | 1.3 |
| 25.028 | 3.55510 | 2.4 |
| 25.819 | 3.44792 | 2.3 |
| 26.415 | 3.37141 | 6.5 |
| 26.743 | 3.33087 | 6.3 |
| 27.708 | 3.21699 | 1.0 |
| 28.979 | 3.07872 | 2.1 |
| 30.466 | 2.93177 | 1.0 |
| 30.661 | 2.91353 | 1.3 |
| 31.456 | 2.84173 | 1.8 |
| 32.994 | 2.71263 | 1.1 |
| 34.468 | 2.59993 | 1.8 |
| 37.740 | 2.38172 | 1.0 |
| 40.480 | 2.22663 | 2.7 |
| 42.486 | 2.12601 | 2.0 |
| 44.042 | 2.05443 | 1.0 |
| 45.337 | 1.99873 | 1.9 |

According to another embodiment of the present invention there is provided an improved process for the preparation of Levofloxacin hemihydrate which comprises
(i) Converting 2,3,4,5-tetrafluoro benzoic acid to its acid chloride by conventional method to give the diethyl-2,3,4,5-tetrafluoro benzoyl malonate.
(ii) Partially hydrolyzing and decarboxylating the resulting diethyl-2,3,4,5-tetrafluoro benzoyl malonate by conventional methods to give ethyl-2,3,4,5-tetrafluoro benzoyl acetate.
(iii) Converting the ethyl-2,3,4,5-tetrafluoro benzoyl acetate by known methods to ethyl-2-(2,3,4,5-tetrafluoro benzoyl)-3-ethoxy acrylate.
(iv) Condensing the ethyl-2-(2,3,4,5-tetrafluoro benzoyl)-3-ethoxy acrylate obtained in step (iii) with (S)-2-amino-1-propanol in a solvent, to give ethyl-2-(2,3,4,5-tetrafluoro benzoyl)-3-[(1-hydroxyprop-2(S)-yl)amino]acrylate,
(v) Cyclising the resulting ethyl-2-(2,3,4,5-tetrafluoro benzoyl)-3-[(1-hydroxy prop-2(S)-yl)amino]acrylate by conventional methods to give (S)-ethyl-9,10-difluoro-2,3-dihydro-3-methyl-7-oxo-7H-pyrido-[1,2,3-Je]-1,4-benzoxazine-6-carboxylate and,
(vi) further hydrolyzing (S)-ethyl-9,10-difluoro-2,3-dihydro-3-methyl-7-oxo-7H-pyrido-[1,2,3-de]-1,4-benzoxazine-6-carboxylate, obtained in step (v) by known methods to give (S)-9,10-difluoro-2,3-dihydro-3-methyl-7-oxo-7H-pyrido-[1,2,3-fife]-1,4-benzoxazine-6-carboxylic acid (namely Levofloxacin Q-acid),
(vii) converting the Levofloxacin Q-Acid by condensing with N-methyl piperazine by using solvent or without using solvent by any known methods to (S)-9-fluoro-3-methyl-10-(4-methyl-1-piperazinyl)-7-oxo-2,3-dihydro-7H-pyrido[1,2,3-cfe]-1,4-benzoxazine-6-carboxylic acid (namely Levofloxacin technical),
(viii) dissolving levofloxacin technical in aqueous alkaline solution,
(ix) treating the resulting solution with activated carbon at room temperature,
(x) removing the undissolved particulate matter by filtration,
(xi) bringing the pH of the aqueous alkaline levofloxacin solution to neutral using dilute mineral acid,
(xii) removing the precipitated particulate matter by filtration,
(xiii) acidifying the resulting solution,
(xiv) treating the acidified solution with activated carbon at room temperature,
(xv) filtering the undissolved particulate matter by filtration,
(xvi) neutralizing the acidic solution,
(xvii) filtering again to remove any particulate matter present and,
(xviii) extracting the resulting product with chlorinated solvent and concentrating under vacuum using aqueous tetrahydrofuran or in admixture with other organic solvents to get highly pure levofloxacin hemihydrate having single individual impurity not more than 0.1% and free from particulate matter & from the other enantiomer (R-form).

Reaction sequence is shown in the scheme given below.

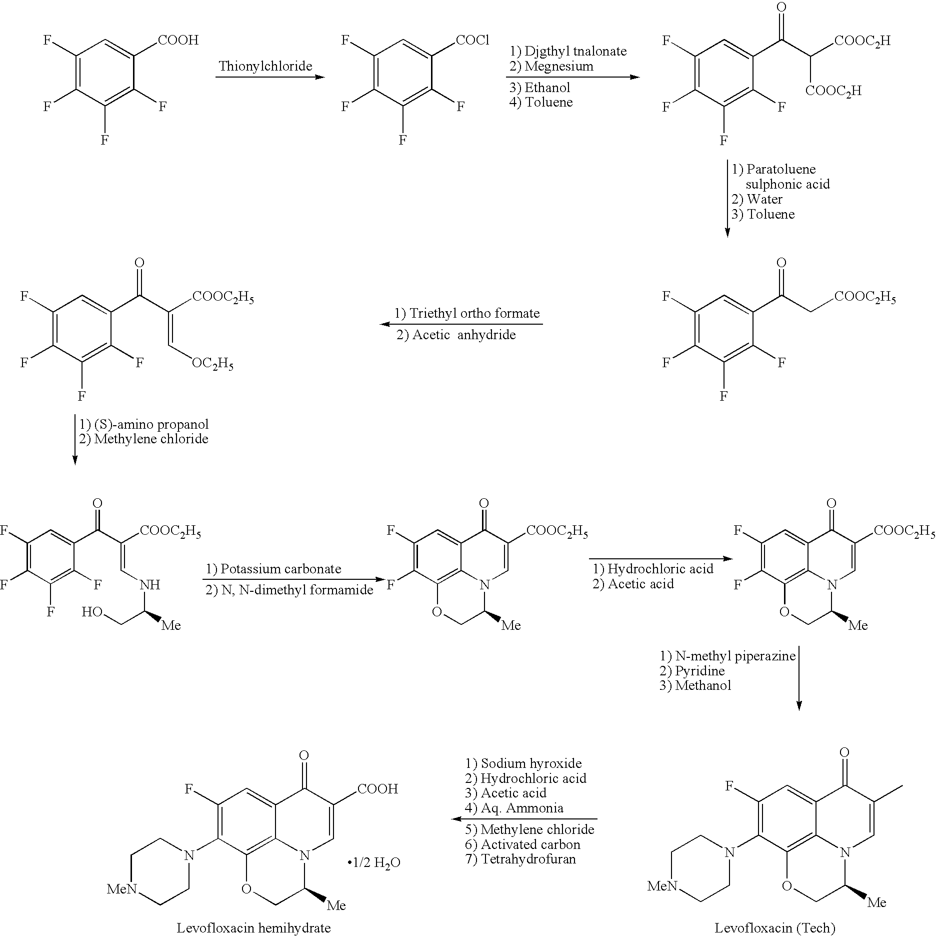

In a preferred embodiment of the invention in step (i) , Diethyl malonate may be acylated using 2,3,4,5-tetrafluoro benzoyl chloride in the presence of magnesium, ethanol by making diethyl ethoxymagnesiomalonate.

The reagents used for the condensation in step (iii) may be triethyl orthoformate and acetic anhydride.

In step (ii) the conversion may be effected using an aqueous medium employing catalytic amount of para toluene sulfonic acid.

An example of the solvent used in step (iv) may be methylene chloride.

The cyclisation in step (v) may be done in the presence of suitable base such as potassium carbonate and an aprotic solvent such as N,N-dimethyl formamide.

The hydrolysis in step (vi) may be carried out using acetic acid and dilute hydrochloric acid.

The details of the present invention are described in the following examples which are provided only to illustrate the invention and therefore should not be construed to limit the scope of the invention.

### EXAMPLE-1

### Preparation of (−)-9-fluoro-3-methyl-10(4-methyl-1-piperazinyl)-7-oxo-2,3-dihydro-7fI-PVrJdO [1,2,,3,-<fel-1.,4-benzoxaziiie-6-carboxylic acid Hemihydrate (Levofloxacin Hemihydrate of the Formula 1)

In a 1000 ml four neck flask fitted with a stirrer, was placed 550 ml of DM water and 50 g of Levofloxacin technical, stir for 15 minutes. Adjusted the pH to 11.0-11.5 with 10% dilute sodium hydroxide at 25-30° C., 2.5 g of activated carbon was added into the flask and stirred for 15 minutes. Filtered the solution, washed the carbon bed with 25 ml of DM water. The pH of the clear filtrate was adjusted to 7.0-7.5 with, 1N Hydrochloric acid at 25-30° C. Filtered the solution and washed the bed with 25 ml of DM water. To the clear filtrate acetic acid was added till the pH reached 4.5-5.0. 2.5 g of activated carbon was added and stirred for 15 minutes. Filtered the solution and again filtered the solution over 0.2 micron Millipore filter paper. To the clear filtrate, aqueous ammonia solution was added to get pH 7.0-7.5. Filtered the solution over 0.2 micron Millipore filter paper. The product was extracted with methylene chloride (3×550 ml). Distilled off combined methylene chloride layer under reduced pressure below 40° C. temperature. Charged 50 ml tetrahydrofuran and co-distilled the methylene chloride. 2-2.5% aqueous tetrahydrofuran (150 ml) was charged into the flask and heated to gentle reflux temperature (58-60° C.). Maintained the reaction mixture under the conditions for 30 minutes, cooled the contents of the flask to 0° C., kept for 1 hour at 0-5° C. Filtered the product and washed the product with chilled 2-2.5% aqueous tetrahydrofuran (50 ml), suck dried for 15 minutes. Dried the product at 70-75° C., to constant weight. The yield was 34.0 g. HPLC purity 99.82% and single individual impurity less than 0.1%. Moisture content: Calculated for Levofloxacin hemihydrate was 2.43% and found is 2.45%. Elemental analysis: calculated for C₁₈H₂₀FN₃O₄. vz H₂O is C 58.37%, H 5.71%, N 11.35% and found is C 58.38%, H 5.67% and N 11.38%.

### EXAMPLE-2

### Preparation of (−)-9-f uoro-3-methyI-10(4-methyl-1-piperazinyl)-7-oxo-2,3-dihyd ro-7fl-pyrido [1,2,3,-<fel-1,4-benzoxazine-6carboxylic acid Hemihydrate (Levofloxa-cin Hemihydrate of the Formula 1)

In a 500 ml four neck flask fitted with a stirrer, was placed 244 ml of DM water and 2.2 g of Levofloxacin technical, stirred for 15 minutes. The pH was adjusted to 11.0-11.5 with 10% dilute sodium hydroxide at 25-30° C., 1.1 g of activated carbon was added into the flask and stirred for 15 minutes. Filtered the solution over 0.2 micron millipore filter paper, washed the carbon bed with 10 ml of DM water. To the clear filtrate, 1N

Hydrochloric acid (54 ml) was added till the pH reached 7.0-7.5 at 25-30° C. Filtered the solution and washed the bed with 10 ml of DM water. To the clear filtrate, added acetic acid (5.3 ml) till pH was reached 4.5-5.0. Added 1.1 g of activated carbon and stirred for 15 minutes. Filtered the solution and again filtered the solution over 0.2 micron Millipore filter paper. To the clear filtrate, aqueous ammonia solution (8.5 ml) was added till the pH was reached 7.0-7.5. Filtered the solution over 0.2 micron Millipore filter paper. The product was extracted with methylene chloride (3×242 ml). Distilled off combined methylene chloride layer under reduced pressure below 40° C. mass temperature. Charged 22 ml ethyl acetate and co-distill the methylene chloride. 2-2.5% aqueous tetrahydrofuran (22 ml) and ethyl acetate (44 ml) were charged into the flask and heated to gentle reflux (58-60° C.). Maintained for 30 minutes cooled the contents of the flask to 0° C., kept for 1 hour at 0-5° C. Filtered the product and washed the product with chilled ethyl acetate (22 ml), suck dry for 15 minutes. Dried the product at 70-75° C., to constant weight. The yield of (−)-9-fluoro-3-methyl-10(4-methyl-1-piperazinyl)-7-oxo-2,3-dihydro-7H-pyrido[1,2,3,-cfe]-1,4-benzoxazine-6-carboxylic acid (Levofloxacin hemihydrate of the formula 1) was 18.2 g. HPLC purity 99.87% and single individual impurity less than 0.1%. Moisture content : Calculated for Levofloxacin hemihydrate is 2.43% and found is 2.48%. Elemental analysis: calculated for C₁₈H₂₀FN₃O₄. V₂ H₂I is C 58.37%, H 5.71%, M 11.35% and found is C 58.36%, H 5.70% and N 11.37%.

### EXAMPLE -3

### Preparation of (−)-9-fluoro-3-methyI-10f4-methyl-1-piperazinyI)-7-oxo-2,3-dihydr(>-7//-PVrJdO [1,2,,3<<-<fel-1,4-benzoxazme-6-carboxy üc acid Hemihydrate (Levofloxaein Hemihydrate of the Formula 1) from 2,3,4,5-tetrafluoro benzoic acid

Step (i) Preparation of 2,3,4,5-Tetrafluoro benzoyl chloride;

Into a three necked 2 L round bottom flask equipped with a mechanical stirrer, thermometer socket and a condenser, thionyl chloride 560 ml, (7.713 mol) 2,3,4,5-Tetrafluoro benzoic acid 200 g (2.577 mol) and N,N-dimethyl formamide 10 ml were charged. The contents of the flask were heated gradually and very cautiously to 85-90° C. during 6 to 8 hrs under reflux. After attaining the mass temperature 85-90° C., a sample was taken out and checked for the content of 2,3,4,5-tetrafluoro benzoic acid by TLC. The contents of the flask were cooled to 45-50° C. Vacuum (600-670 mm/Hg) was applied and thionyl chloride was distilled off up to reaction mass temperature 90° C. The contents of the flask was cooled to room temperature and break the vacuum with nitrogen. High vacuum (690-720 mm/Hg) was applied to the contents of flask and collected I^{st} fraction below 70° C. vapour temperature. Pure fraction was collected separately vapour temperature above 70° C. Yield 530 g (96.8%). HPLC purity 98.50%

Step (ii) Preparation of Diethyl-2,3,4,5-Tetrafluoro benzoyl malonate

Into a four neck 10 Lit round bottom flask equipped with a mechanical stirrer, thermometer socket and a condenser, 3.4 L toluene, 565 g (3.53 mol) of diethyl malonate and 365 ml of ethanol were charged. The contents of the flask were stirred at room temperature for 20 min. The moisture content of the contents of flask was checked. It should be not more than 0.1% w/v. The contents of the flask were transferred into another single neck flask. Into the four necked flask, 150 ml of ethanol, 86 g (3.54 mol) magnesium turnings and catalytic amount of chloroform (10 ml) were charged and waited for 30 min. for initiation of the reaction. The above prepared diethyl malonate, ethanol and toluene mixture was gradually and uniformly added to reaction mixture while stirring at 70-90° C. (exothermic reaction) over a period of 3 to 4 hrs. The contents of the flask were stirred for additional 2 hrs. at 70-90° C. and cooled to 50-55° C. and distilled off solvent under vacuum (600-650 mm of Hg). The residual ethanol was removed by azeotropic distillation with toluene, then residue is redissolved in 2 L of dry toluene and 200 ml of tetrahydrofuran and cooled to 30-35° C. The contents of the flask are further cooled to 0-5° C. and 500 g (2.35 mol) 2,3,4,5-Tetrafluoro benzoyl chloride with 500 ml toluene mixture solution is added slowly drop wise over a period of 1-2 hrs. at 0-5° C. The contents of the flask are stirred for 30 min. at 0-5° C. and allowed to 20-25° C. and maintained for 30 min at 20-25 ° C. TLC is checked for the content of 2,3,4,5-Tetrafluoro benzoic acid. Dilute sulfuric acid (sulfuric acid content 11.5-12.5% w/v) was added to the contents of the flask below 35° C. The contents of the flask were stirred for 15 min. at 25-30° C. and separated both layers. The aqueous layer was extracted with toluene (2×500 ml) twice. Organic layers were mixed and washed with vacuum salt solution (200 g vacuum salt with 1 L water). The organic layer was filtered over high flow bed and again washed with vacuum salt solution (200 g of vacuum salt with 1 L DM water) followed by DM water (500 ml) washing. The aqueous layer of DM water washing was checked for sulfate test (sulfates should be absent). If sulfates were present, again the organic layer was washed with DM water (500 ml) till sulfates were absent. Organic layer was concentrated under vacuum (550-650 mm/Hg) up to mass temperature 80° C. to give quantitative yield of diethyl-2,3,4,5-tetrafluoro benzoyl malonate. HPLC purity: 98.35%.

Step (iii) Preparation of (−)-Ethyl-9q θ-difluoro-23-dihydro-3-methyl-7-oxo-7.ff-pyrido-fl,,2,3-<fel-1.,4-benzoxazine-6-carboxylate:

Into a 3 lit 3 neck round bottom flask is provided with a mechanical stirrer, thermometer socket and a condenser, DM water (1265 ml) diethyl-2₅3,4,5-tetrafluoro benzoyl malonate 790.58 g (2.35 mol) and para toluene sulphonic acid (3.4 g) were charged. The contents of the flask were refluxed (80-90° C.) under stirring and maintained at the same temperature for 3 hrs. After completion of 3 hrs. maintenance, TLC was checked for ketodiester content. If ketodiester content is more than 1.0%, then para toluene sulphonic acid (3.4 g) was charged into the reaction mixture and maintained 3 hrs. at reflux temperature. TLC was checked for ketodiester content present in the reaction mixture. If ketodiester content was more than 1.0% then again para toluene sulphonic acid (3.4 g) was charged and refluxed for 3 hrs. same thing was repeated till TLC shows the content of ketodiester was below 1.0%. The reaction mixture was cooled to 30-35° C. and separated both layers. Aqueous layer was extracted with toluene (2×500 ml) twice. Organic layers were mixed and washed with 5% sodium bicarbonate solution (500 ml) following by DM water (2×500 ml) twice. The aqueous layer of second washing was checked for sulfate test. If sulfates present the organic layer was again washed with DM water (500 ml) till sulfates absent. Organic layer was concentrated under vacuum (650-750 mm/Hg) at 60-65° C. to give 608 g of ethyl-2,3,4,5-tetrafluorobenzoyl acetate (ketoester). To ketoester without further purification, 544 ml (5.76 mol) Acetic anhydride and 511 g (3.45 mol) triethyl orthoformate were charged into above reaction mixture. The contents of the RB flask were heated to 120-125° C. and maintained for 4 hrs. at 120-125° C., low boilers were collected at receiver end while maintaining the reaction at 120-125° C. After 4 hrs maintenance, the contents of the RJB flask was cooled to 90-100° C., vacuum (650-700 mm/Hg) was applied to the reaction mixture and solvent was distilled off up to mass temperature 125° C. The reaction mixture was cooled to 80-85° C. and 250 ml toluene was charged. Again vacuum (650-700 mm/Hg) was applied and solvent was distilled off up to 125° C. The distillation was continued at 125° C. till distillation ceases. The reaction mixture was cooled to 30-35° C. 800 ml methylene chloride was charged and further cooled to 0-5° C. while stirring. 133 g (1.77 mol) (S)-(+)-2-amino-1-propanol with 270 ml methylene chloride was charged into reaction mixture at 0-5° C. over a period of 1-2 hrs. After addition, reaction mixture was allowed to 30-35° C. gradually and maintained at 30-35° C. for 2 hrs. Reaction mixture was washed with water twice and dried with anhydrous sodium sulfate. Meanwhile 850 ml N-dimethyl formamide, 444 g potassium carbonate were charged into another flask provided with mechanical stirrer and distillation set-up. The contents were stirred and heated to 120° C. The methylene chloride layer was added during 2 hrs. at 120° C. and methylene chloride was collected at receiver end. Stirring was continued for another 30 minutes the reaction mixture was cooled to 65-70° C. Vacuum (650-700 mm of Hg) was applied to the reaction mixture and distilled off the solvent till half quantity of charged N,N-dimethyl formamide was collected. The reaction mixture was cooled to 20-25° C. DM water (4 L) was charged into the reaction mixture while stirring. Stirring was continued for 1 hr. at 25-30° C. and filtered. Material was washed with DM water (500 ml) and suck dried. 650 ml acetone and wet material were charged into the flask. The contents of the flask were cooled to 0-5° C. and maintained 30 minutes at 0-5° C. Material was filtered and washed with 650 ml acetone. The material was dried at 70-75° C. to give 315 g (−)-ethyl-9,10-difluoro-2,3-dihydro-3-methyl-7-oxo-7H-pyrido-[1,2,3-de]-1,4-benzoxazine-6-carboxylate. HPLC purity: 99.23°/\dot{o}.

Step (iv) Preparation of MˆJO-DifluoroˆJ-dihydroˆ-mett.Lylˆ-oxo-Tff-pyrido-fl.2,3-de 1-1,4-benzoxazine-6-carboxylic acid:

Into a 1 L 3 neck round bottom flask equipped with mechanical stirrer, thermometer socket and condenser, 125 ml water, 125 ml concentrated hydrochloric acid, 250 ml acetic acid and 100 g (0.32 mol) (−)-ethyl-9,10-difluoro-2,3-dihydro-3-methyl-7-oxo-7H pyrido [1,2,3-cfe]-1,4-benzoxazine-6-carboxylate were charged. The contents were heated to 75-80° C. and maintained for 6 hrs at 75-80° C. After 6 hrs the conversion of reaction was monitored by TLC. The reaction mixture was cooled to 15-20° C. and maintained for 1 hr. at 15-20° C. Material was filtered and washed with water. Wet material was slurred with water at 45-50° C. and filtered. Dried at 70-80° C. to give 80 g of (−)9,10-difluoro-2,3-dihydro-3-methyl-7-oxo-7H-pyrido[1,2,3-de]-1,4-benzoxazine-6-carboxylic acid. HPLC purity: 99.33%.

Step (v) Preparation of (-V9-fluoro-3-methyl-10(4-methyl-1-piperazinvIV7-oxo-2,3-dihydro-7//-pyrido ri,2,3,-de1-1,4-benzoxazine-6-carboxylic acid (Levofloxacin Technical):

Into a 3 necked round bottom flask equipped with a mechanical stirrer, thermometer socket and a condenser, 228 g (2.28 mol) N-methyl piperazine, 1.2 L pyridine and 200 g (0.712 mol) (−)-9,10-difluoro-2,3-dihydro-3-methyl-7-oxo-7H-pyrido[1,2,3-de]-1,4-benzoxazine-6-carboxylic acid are charged the contents are heated to reflux at 120° C. for 10 hrs. and conversion is monitored by TLC. The reaction mixture is concentrated under vacuum (600-650 mm of Hg) to leave residue, 200 ml methanol is charged and further concentrated. 800 ml Methanol is charged to residue and heated to reflux for 30 min. reaction mass is cooled to 10-15° C. and maintained for 1 hour, material is filtered and washed with 100 ml chilled methanol to give 210 g of Levofloxacin (technical) HPLC purity: 98.58%.

Step (vi) Preparation of (−)-9-fluoro-3-methyl-10(4-methyl-1-piperazinyl)-7-oxo-2,3-dihydro-7Ir-pyrido fl,2,3,-<fel-1-4-benzoxazine-6-carboxylic acid hemihycMrate (Levofloxacin Hemihydrate of the Formula 1);

In a 4 neck flask fitted with a stirrer, was placed 2.2 L of DM water and 200 g of Levofloxacin technical, stir for 15 minutes. Adjusted the pH to 11.0-11.5 with 10% dilute sodium hydroxide at 25-30° C., 10 g of activated carbon was added into the flask and stirred for 15 minutes. Filtered the solution, washed the carbon bed with 100 ml of DM water. The pH of the clear filtrate was adjusted to 7.0-7.5 with, 1N Hydrochloric acid at 25-30° C. Filtered the solution and washed the bed with 100 ml of DM water. To the clear filtrate acetic acid was added till the pH reached 4.5-5.0. 10 g of activated carbon was added and stirred for 15 minutes. Filtered the solution and again filtered the solution over 0.2 micron Millipore filter paper. To the clear filtrate, aqueous ammonia solution was added to get pH 7.0-7.5. Filtered the solution over 0.2 micron Millipore filter paper. The product was extracted with methylene chloride (3×2.2 L). Distilled off combined methylene chloride layer under reduced pressure below 40° C. temperature. Charged 200 ml tetrahydrofuran and co-distilled the methylene chloride. 2-2.5% aqueous tetrahydrofuran (600 ml) was charged into the flask and heated to gentle reflux temperature (58-60° C.). Maintained the reaction mixture under the conditions for 30 minutes, cooled the contents of the flask to 0° C., kept for 1 hour at 0-5° C. Filtered the product and washed the product with chilled 2-2.5% aqueous tetrahydrofuran (200>ml), suck dried for 15 minutes. Dried the product at 70-75° C., to constant weight. The yield was 142 g. HPLC purity 99.90% and single individual impurity less than 0.1%. Moisture content: Calculated for Levofloxacin hemihydrate was 2.43% and found is 2.50%. Elemental analysis: calculated for C₁₈H₂₀FN₃O₄.V₂H₂O is C 58.37%, H 5.71%, N 11.35% and found is C 58.35%, H 5.72% and N 11.38%.

Advantages of the Present Invention

(i) Levofloxacin hemihydrate prepared having single individual impurity less than 0.1% and free from particulate matter.
(ii) Levofloxacin hemihydrate prepared is free from the other enantiomer (R-form).
(iii) The process of the present invention provides industrially feasible process.
(iv) The process is simple and safe and environmentally friendly

It should be noted that the invention also envisages other embodiments falling within the scope of the present invention which will be obvious and apparent to one skilled in the art from the foregoing disclosure.

1. An improved process for preparation of Levofloxacin hemihydrate having single individual impurity not more than 0.1% and free from particulate matter and from the other enantiomer (R-form) which comprises
i. dissolving levofloxacin technical grade in aqueous alkaline solution,
ii. treating the resulting solution with activated carbon at room temperature,
iii. removing the undissolved particulate matter filtration,
iv. bringing the pH of the aqueous alkaline levofloxacin solution to neutral using dilute mineral acid,
v. removing the precipitated particulate matter by filtration,
vi. acidifying the resulting solution,
vii. treating the acidified solution with activated carbon at room temperature,
viii. filtering the undissolved particulate matter by filtration,
iv. neutralizing the acidic solution,
x. filtering again to remove any particulate matter present and,
xi. extracting the resulting product with chlorinated solvent and concentrating under vacuum using aqueous tetrahydrofuran or in admixture with other organic solvents to get highly pure levofloxacin hemihydrate having single individual impurity less than 0.1% and free from particulate matter and from the other enantiomer (R-form).

2. An improved process as claimed in claim 1 wherein the filtration in steps (viii) and (x) may be effected using a 0.2 micron filter.

3. An improved process as claimed in claim 1 wherein the alkaline Levofloxacin solution is stirred at a pH in the range of 8.0 to 12.0, preferably 10.0-12.0, more preferably 11.0-11.5.

4. An improved process as claimed in claim 1 wherein the alkali used is selected from sodium hydroxide or potassium hydroxide, preferably sodium hydroxide and the concentration of the solution is 5 to 20% preferably 8-10%.

5. An improved process as claimed in claim 1 wherein the pH is brought to 7.0-7.5 using dilute hydrochloric acid, preferably 0.5N to 5N hydrochloric acid, more preferably 1N hydrochloric acid.

6. An improved process as claimed in claim 1 wherein the precipitated particulate matter is filtered and the pH is adjusted to 3.0-6.0 preferably 4.0 to 5.5 more preferably 4.5-5.0 using glacial acetic acid.

7. An improved process as claimed in claim 1 wherein the aqueous acidic levofloxacin solution is treated with activated carbon at room temperature and the clear solution is filtered the pH to neutral preferably 7.0-7.5 using dilute aqueous ammonia solution.

8. An improved process as claimed in claim 1 wherein the neutral aqueous solution is again filtered and extracted with chlorinated solvent preferably methylene chloride.

9. An improved process as claimed in claim 1 wherein the extract is concentrated under vacuum (600-650 mm of Hg) below 40° C. and the resulting residue is concentrated after stirring with tetrahydrofuran or its mixture with any other another organic solvent.

10. An improved process as claimed in claim 1 wherein the residue is slurred with 1-5% aqueous tetrahydrofuran preferably with 2-2.5% aqueous tetrahydrofuran.

11. An improved process as claimed in claim 1 wherein the slurring with tetrahydrofuran is effected at 40-70° C. preferably at 50-60° C. more preferably at 58-60° C.

12. An improved process as claimed in claim 1 wherein the slurring with tetrahydrofuran is effected for a period in the range of 30 minutes to 2 hours preferably 30 minutes to 1 hour and then cooled to −5 to 15° C. preferably 0-5° C. and stirred for 30 minutes to 2 hours preferably 1 hour to 1 hour 30 minutes.

13. An improved process as claimed in claim 1 wherein the product is filtered and suck dried for 15 minutes to 1 hour preferably 30 minutes to 45 minutes and the product was dried at 50-80° C. preferably at 70-75° C. for 2 to 7 hours preferably 4-6 hours more preferably 5 to 5 hours 30 minutes.

14. An improved process for the preparation of Levofloxacin hemihydrate which comprises,
i. Converting 2,3,4,5-tetrafluoro benzoic acid to its acid chloride by conventional method to give the diethyl-2,3,4,5-tetrafluoro benzoyl malonate,
ii. Partially hydrolyzing and decarboxylating the resulting diethyl-2,3,4,5-tetrafluoro benzoyl malonate by conventional methods to give ethyl-2,3,4,5-tetrafluoro benzoyl acetate,
iii. Converting the ethyl-2,3,4,5-tetrafluoro benzoyl acetate by known methods to ethyl-2-(2,3,4,5-tetrafluoro benzoyl)-3-ethoxy acrylate,
iv. Condensing the ethyl-2-(2,3,4,5-tetrafluoro benzoyl)-3-ethoxy acrylate obtained in step (iii) with (S)-2-amino-1-propanol in a solvent, to give ethyl-2-(2,3,4,5-tetrafluorobenzoyl)-3-[(1-hydroxyprop-2(S)-yl)amino]acrylate,
v. Cyclising the resulting ethyl-2-(2,3,4,5-tetrafluorobenzoyl)-3-[(1-hydroxyprop-2(S)-yl)amino]acrylate by conventional methods to give (S)-ethyl-9,10-difluoro-2,3-dihydro-3-methyl-7-oxo-7H-pyrido-[12,3-de]-1,4-benzoxazine-6-carboxylate and,
vi. further hydrolyzing (S)-ethyl-9,10-difluoro-2,3-dihydro-3-methyl-7-oxo-7H-pyrido-[1,2,3-de]-1,4-benzoxazine-6-carboxylate, obtained in step (v) by known methods to give (S)-9,10-difluoro-2,3-dihydro-3-methyl-7-oxo-7H-pyrido-[1,2,3-de]-1,4-benzoxazine-6-carboxylic acid (namely Levofloxacin Q-acid),
vii. converting the Levofloxacin Q-Acid by condensing with N-methyl piperazine by using solvent or without using solvent by any known methods to (S)-9-fluoro-3-methyl-10-(4-methyl-1-piperazinyl)-7-oxo-2,3-dihydro-7H-pyrido[1,2,3-de]-1,4-benzoxazine-6-carboxylic acid (namely Levofloxacin technical),
viii. dissolving levofloxacin technical in aqueous alkaline solution,
ix. treating the resulting solution with activated carbon at room temperature,
x. removing the undissolved particulate matter by filtration,
xi. bringing the pH of the aqueous alkaline levofloxacin solution to neutral using dilute mineral acid,
xii. removing the precipitated particulate matter by filtration,
xiii. acidifying the resulting solution,
xiv. treating the acidified solution with activated carbon at room temperature,
xv. filtering the undissolved particulate matter by filtration,
xvi. neutralizing the acidic solution,
xvii. filtering again to remove any particulate matter present and,
xviii. extracting the resulting product with chlorinated solvent and concentrating under vacuum using aqueous tetrahydrofuran or in admixture with other organic solvents to get highly pure levofloxacin hemihydrate having single individual impurity not more than 0.1% and free from particulate matter and from the other enantiomer (R-form).

15. An improved process as claimed in claim 14 wherein in step (i), Diethyl malonate is acylated using 2,3,4,5-tetrafluoro benzoyl chloride in the presence of magnesium, ethanol by making diethyl ethoxymagnesiomalonate.

16. An improved process as claimed in claim 14 wherein in step (ii) the conversion is effected using an aqueous medium employing catalytic amount of para toluene sulfonic acid.

17. An improved process as claimed in claim 14 wherein in the reagents used for the condensation in step (iii) is triethyl orthoformate and acetic anhydride.

18. An improved process as claimed in claim 14 wherein the solvent used in step (iv) is methylene chloride.

19. An improved process as claimed in claim 14 wherein the cyclisation in step (v) is done in the presence of suitable base such as potassium carbonate and an aprotic solvent such as N,N-dimethyl formamide.

20. An improved process as claimed in claim 14 wherein the hydrolysis in step (vi) is carried out using acetic acid and dilute hydrochloric acid.

21. An improved process as claimed in claim 14 wherein, the condensation in step (vii) is carried out either using aprotic solvent or without using solvent.

22. An improved process as claimed in claim 21 wherein, the aprotic solvent which is used for condensation is pyridine.

23. An improved process as claimed in claim 14 wherein the filtration in steps (xv) and (xvii) may be effected using a 0.2 micron filter.

24. An improved process as claimed in claim 14 wherein the alkaline Levofloxacin solution is stirred at a pH in the range of 8.0 to 12.0, preferably 10.0-12.0, more preferably 11.0-11.5.

25. An improved process as claimed in claim 14 wherein the alkali used in step (viii) is selected from sodium hydroxide or potassium hydroxide, preferably sodium hydroxide and the concentration of the solution is 5 to 20% preferably 8-10%.

26. An improved process as claimed in claim 14 wherein the pH is brought to 7.0-7.5 using dilute hydrochloric acid, preferably 0.5N to 5N hydrochloric acid, more preferably 1N hydrochloric acid.

27. An improved process as claimed in claim 14 wherein the precipitated particulate matter is filtered and the pH is adjusted to 3.0-6.0 preferably 4.0 to 5.5 more preferably 4.5-5.0 using glacial acetic acid.

28. An improved process as claimed in claim 14 wherein the aqueous acidic levofloxacin solution is treated with activated carbon at room temperature and the clear solution is filtered the pH to neutral preferably 7.0-7.5 using dilute aqueous ammonia solution.

29. An improved process as claimed in claim 14 wherein the neutral aqueous solution is again filtered and extracted with chlorinated solvent preferably methylene chloride.

30. An improved process as claimed in claim 14 wherein the extract is concentrated under vacuum (600-650 mm of Hg) below 40° C. and the resulting residue is concentrated after stirring with tetrahydrofuran or its mixture with another organic solvent.

31. An improved process as claimed in claim 14 wherein the residue is slurred with 1-5% aqueous tetrahydrofuran preferably with 2-2.5% aqueous tetrahydrofuran.

32. An improved process as claimed in claim 14 wherein the slurring with tetrahydrofuran is effected at 40-70° C. preferably at 50-60° C. more preferably at 58-60° C.

33. An improved process as claimed in claim 14 wherein the slurring with tetrahydrofuran is effected for a period in the range of 30 minutes to 2 hours preferably 30 minutes to 1 hour and then cooled to −5 to 15° C. preferably 0-5° C. and stirred for 30 minutes to 2 hours preferably 1 hour to 1 hour 30 minutes.

34. An improved process as claimed in claim 14 wherein the product is filtered and suck dried for 15 minutes to 1 hour preferably 30 minutes to 45 minutes and the product was dried at 50-80° C. preferably at 70-75° C. for 2 to 7 hours preferably 4-6 hours more preferably 5 to 5 hours 30 minutes.

35. An improved process for the preparation of Levofloxacin hemihydrate having single individual impurity not more than 0.1% and free from particulate matter and from the other enantiomer (R-form) from Levofloxacin technical.

36. An improved process for the preparation of Levofloxacin hemihydrate having single individual impurity not more than 0.1% and free from particulate matter and from the other enantiomer (R-form) from 2,3,4,5-tetrafluoro benzoic acid.
